(19) United States
(12) Patent Application Publication
(10) Pub. No.: US 2017/0262576 A1
(43) Pub. Date: Sep. 14, 2017

(54) SYSTEM, METHOD AND SOFTWARE FOR ANALYSIS OF INTRACELLULAR SIGNALING PATHWAY ACTIVATION USING TRANSCRIPTOMIC DATA

(71) Applicant: Canada Cancer and Aging Research Laboratories Inc., Lethbridge AB, CA (US)

(72) Inventors: ANTON BUZDIN, ISTRA (RU); NICOLAY BORISOV, KRASNOZNAMENSK (RU); ALEXANDER ZHAVORONKOV, MOSCOW (RU)

(21) Appl. No.: 15/125,487

(22) PCT Filed: Mar. 2, 2015
(86) PCT No.: PCT/IL2015/000011

§ 371 Date: Sep. 12, 2016

Related U.S. Application Data
(60) Provisional application No. 61/952,400, filed on Mar. 13, 2014.

(51) Int. Cl. G06F 19/12; C12Q 1/68

(52) U.S. Cl. CPC G06F 19/12; C12Q 1/6809; C12Q 1/6886; C12Q 2600/158

(57) ABSTRACT

The present invention provides systems, methods and software for analysis of the intracellular signaling pathway activation (SPA), the method including analyzing activator and repressor roles of a plurality of gene expression gene products in a plurality of pathways in a sample of a subject to determine a pathway activation strength (PAS) for each of the plurality of pathways and comparing the pathway activation strength (PAS) in at least one sick subject with at least one healthy subject to determine intracellular signaling pathway activation (SPA) associated with a disease or disorder in the at least one sick subject.

### FIELD OF THE INVENTION

The present invention relates generally to systems and methods of analysis of transcriptomic data, and more specifically to systems and methods for intracellular signaling pathway activation using transcriptomic data.

### BACKGROUND OF THE INVENTION

In the twentieth century, enormous strides were made in combating infectious diseases, in their detection and drugs to treat them. The major problem in the medical world has thus shifted from treating acute diseases to treating chronic diseases. Over the last few decades, with the advent of genetic engineering, much research and funding has been invested in genomics and gene-based personalized medicine. A need has arisen to develop diagnostic tools for use in the characterization of personalized aspects of chronic diseases.

Intracellular signaling pathways (SPs) regulate numerous processes involved in normal and pathological conditions including development, growth, aging and cancer. Many bioinformatic tools have been developed, which analyze SPs.

The information relating to signaling pathway activation (SPA) can be obtained from the massive proteomic or transcriptomic data. Although the proteomic level may be somewhat closer to the biological function of SPA, the transcriptomic level of studies today is far more feasible in terms of performing experimental tests and analyzing the data.

Intracellular signaling pathways (SPs) regulate numerous processes involved in normal and pathological conditions including development, growth, aging and cancer. Many bioinformatic tools have been developed recently that analyze SPs. However, none of them makes it possible to efficiently do the high-throughput quantification of pathway activation scores for the individual biological samples. Here we propose a method for quick, informative and large-scale screening of changes in signaling pathway activation (SPA) in cells and tissues. These changes may reflect various differential conditions like differences in physiological state, aging, disease, treatment with drugs, infections, media composition, additives, etc. One of the potential applications of SPA studies may be in utilizing mathematical algorithms to identify and rank the medicines based on their predicted efficacy.

The information about SPA can be obtained from the massive proteomic or transcriptomic data. Although the proteomic level may be somewhat closer to the biological function of SPA, the transcriptomic level of studies today is far more feasible in terms of performing experimental tests and analyzing the data. The transcriptomic methods like Next-generation sequencing (NGS) or microarray analysis of RNA can routinely determine expression levels for all or virtually all human genes (Shirane, 2004). Transcriptome profiling may be performed for the minute amount of the tissue sample, not necessarily fresh, but also for the clinical formalin-fixed, paraffin-embedded (FFPE) tissue blocks. For the molecular analysis of cancer, gene expression can be interpreted in terms of abnormal SPA features of various pro- and antimitotic signaling pathways. Such analysis may improve further decision-making process of treatment strategy selection by the clinician.

Pro- and antimitotic SPs that determine various stages of cell cycle progression remained in the spotlight of the computational biologists for more than a decade (Kholodenko, 1999; Borisov, 2009; Kuzmina, 2011). Today, hundreds of SPs and related gene product interaction maps that show sophisticated relationships between the individual molecules, are catalogued in various databases like UniProt (The UniProt consortium, 2011), HPRD (Mathivanan, 2006), QIAGEN SABiosciences (SABiosciences), WikiPathways (Bauer-Mehren, 2009), Ariadne Pathway Studio (Nikitin, 2004), SPIKE (Elkon, 2008), Reactome (Haw, 2012), KEGG (Nakaya, 2013), etc.

One group of bioinformatic approaches integrated the analysis of transcriptome-wide data with the models employing the mass action law and Michaelis-Meten kinetics (Yizhak, 2013). These methods which were developing during last fifteen years, however, remained purely fundamental until recently, primarily, because of the multiplicity of interaction domains in the signal transducer proteins that enormously increase the interactome complexity (Borisov, 2008; Conzelman, 2006). Secondly, a considerable number of unknown free parameters, such as kinetics constants and/or concentrations of protein molecules, significantly complicated the SPA analysis. Yizhak et al. (2013) suggested that the clinical efficiency of several drugs, e.g. geroprotectors, may be evaluated as the ability to induce the kinetic models of the pathways into the steady state. However, protein-protein interactions were quantitatively characterized in detail only for a tiny fraction of SPs. This approach is also time-consuming since to process each transcriptomic dataset it requires extensive calculations for the kinetic models (Yizhak, 2013).

However, all the contemporary bioinformatical methods that were proposed for digesting large-scale gene expression data followed by recognition and analysis of SPs, have an important disadvantage. They do not allow tracing the overall pathway activation signatures and quantitively estimate the extent of SPA (Hwang, 2012; Kuzmina, 2011; Yizhak, 2013). This may be due to lack of the definition of the specific roles of the individual gene products in the overall signal transduction process, incorporated in the calculation matrix used to estimate SPA.

US2008254497A provides a method of determining whether tumor cells or tissue is responsive to treatment with an ErbB pathway-specific drug. In accordance with the invention, measurements are made on such cells or tissues to determine values for total ErbB receptors of one or more types, ErbB receptor dimers of one or more types and their phosphorylation states, and/or one or more ErbB signaling pathway effector proteins and their phosphorylation states. These quantities, or a response index based on them, are positively or negatively correlated with cell or tissue responsiveness to treatment with an ErbB pathway-specific drug. In one aspect, such correlations are determined from a model of the mechanism of action of a ErbB pathway-specific drug on an ErbB pathway. Preferably, methods of the invention are implemented by using sets of binding compounds having releasable molecular tags that are specific for multiple components of one or more complexes formed in ErbB pathway activation. After binding, molecular tags are released and separated from the assay mixture for analysis.

U.S. Pat. No. 8,623,592 discloses methods for treating patients which methods comprise methods for predicting responses of cells, such as tumor cells, to treatment with therapeutic agents. These methods involve measuring, in a sample of the cells, levels of one or more components of a cellular network and then computing a Network Activation State (NAS) or a Network Inhibition State (NIS) for the cells using a computational model of the cellular network. The response of the cells to treatment is then predicted based on the NAS or NIS value that has been computed. The invention also comprises predictive methods for cellular responsiveness in which computation of a NAS or NIS value for the cells (e.g., tumor cells) is combined with use of a statistical classification algorithm. Biomarkers for predicting responsiveness to treatment with a therapeutic agent that targets a component within the ErbB signaling pathway are also provided.

There thus remains a need for systems and methods, which provide rapid personalized analyses of signaling pathway activation, based upon small tissue samples from an individual. These systems need to be applied to provide predictions of disease or disorder diagnosis and disease or disorder progress.

### SUMMARY OF THE INVENTION

It is an object of some aspects of the present invention to provide systems and methods, which provide rapid personalized analyses of signaling pathway activation, based upon small tissue samples from an individual.

The present invention provides novel methods and systems for determining biological pathway activation, repression and inhibition. The system, called “OncoFinder™”, is constructed and configured for both quantitative and qualitative analysis of the intracellular signaling pathway activation (SPA). This method is universal and may be used for the analysis of any physiological, stress, malignancy and other perturbed conditions at the molecular level. In contrast to the other existing techniques for aggregation and generalization of the gene expression data for individual samples, the methods of the present invention, disclosed herein provide ways to distinguish the positive/activator and negative/repressor role of every gene product in each pathway.

The present invention discloses that the relative importance of each gene product in a pathway can be assessed using kinetic models for “low-level” protein interactions. Although the importance factors for the pathway members cannot be so far established for most of the signaling pathways due to the lack of the required experimental data, it is shown that ignoring these factors can be sometimes acceptable and that the simplified formula for SPA evaluation may be applied for many cases.

There is thus provided according to an embodiment of the present invention, a method for determining pathway activation in a biological sample, the method including;
a. determining for a biological pathway at least one concentration of a plurality of gene products involved in the pathway of the biological sample; and
b. comparing the at least one concentration of the plurality of the gene product of the biological sample with a control set of concentrations of same gene products of at least one control sample to determine the pathway activation in the biological sample.

Additionally, according to an embodiment of the present invention, the biological sample is selected from the group consisting of a tissue sample, a cell culture, an individual single cell, a bodily sample, an organism sample and a microorganism sample.

Furthermore, according to an embodiment of the present invention, the pathway activation is a biological pathway activation.

Moreover, according to an embodiment of the present invention, the biological pathway is a signaling pathway.

Further, according to an embodiment of the present invention, the biological pathway is a metabolic pathway.

Yet further, according to an embodiment of the present invention, the plurality of gene products includes a set of at least five gene products.

Additionally, according to an embodiment of the present invention, the method further includes;
c. calculating a pathway activation strength (PAS), indicative of the pathway activation of the biological pathway.

Additionally, according to an embodiment of the present invention, the calculating step includes adding concentrations of the set of the at least five gene products of the sample and comparing to a same set in the at least one control sample.

According to another embodiment of the present invention, the gene products provide at least one function in the biological pathway.

Additionally, according to an embodiment of the present invention, the at least one function includes an activation function and a suppressor function.

According to another embodiment of the present invention, the at least one function includes an up-regulating function and a down-regulating function.

Moreover, according to an embodiment of the present invention, the method further includes determining an overall signal outcome (SO).

Additionally, according to an embodiment of the present invention, the signal outcome (SO) is defined as

$$S O = \frac{{\prod }_{i = 1}^{N} {[ AGEL ]}_{i}}{{\prod }_{j = 1}^{M} {[ RGEL ]}_{j}} ,$$

and wherein multiplication is performed for possible activator and repressor proteins in the pathway, and [AGEL]ᵢ and [RGEL]ⱼ are relative gene expression levels of activator (i) and repressor (j) members, respectively.

Furthermore, according to an embodiment of the present invention, the determining step includes at least one of profiling gene expression, RNA profiling, RNA sequencing, DNA profiling, DNA sequencing, protein profiling, amino acid sequencing, at least one immunochemical methodology, a mass spectrometry analysis, a microarray technology, a quantitative PCR methodology and combinations thereof.

Further, according to an embodiment of the present invention, the method is quantitative. Additionally or alternatively, the method is qualitative.

Additionally, according to an embodiment of the present invention, the biological sample is from a vertebrate subject.

According to an embodiment of the present invention, the subject is mammalian.

Furthermore, according to an embodiment of the present invention, the subject is human.

Additionally, according to an embodiment of the present invention, the subject is sick.

Additionally, according to an embodiment of the present invention, the sick subject suffers from a proliferative disease or disorder.

Moreover, according to an embodiment of the present invention, the proliferative disease or disorder is cancer.

Furthermore, according to an embodiment of the present invention, the PAS is defined by

$${PAS}_{p} = {\sum }_{n} {ARR}_{np} \cdot lg ( {CNR}_{n} ) .$$

According to an embodiment of the present invention, a signaling pathway activation (SPA) is determined by

$${PAS}_{p}^{( 1 , 2 )} = {\sum }_{n} {ARR}_{np} \cdot {BTIF}_{n} \cdot {w}_{n}^{( 1 , 2 )} \cdot lg ( {CNR}_{n} ) .$$

According to an embodiment of the present invention, pathway activation strength, which is the indicator of the pathway activation, is calculated in an investigated biosample.

According to an embodiment of the present invention, this PAS score depends on the concentrations of the gene products that are involved in the pathway functioning, in the biosample under investigation, normalized to the concentration of the respective gene products in the control set of gene expression data.

According to further embodiments of the present invention PAS is the additive value and it is dependent on the concentrations of all or several (at least five) gene products involved in the pathway functioning.

Moreover, according to further embodiments of the present invention, individual gene product impact in PAS depends on the role played by the gene in the functioning of the pathway. This role is introduced to the formula by the respective coefficients. These coefficients may discriminate multiple roles of the gene products in the pathway, but with a minimum of the two major roles: activator and repressor of the pathway signalization.

The present invention includes analysis of PAS using the specific formulae described herein or any other appropriate formulae. These formulae may include use of one or more formula, multiple formulae, using CNR instead of lg(CNR), adding any other additional coefficients, adding other parameters and the like. Any algorithm for a PAS calculation, including the following four items, are deemed to be part of the present invention:—
a) calculating Pathway activation strength, which is the indicator of the pathway activation in an investigated biosample;
b) calculating the PAS score depending on the concentrations of the gene products that are involved in the pathway functioning, in the biosample under investigation, normalized to the concentration of the respective gene products in the control set of gene expression data;
c) PAS is the additive value and it is dependent on the concentrations of all or several (at least five) gene products involved in the pathway functioning; and
d) Individual gene product impact in PAS depends on the role played by the gene in the functioning of the pathway. This role is introduced to the formula by the respective coefficients. These coefficients may discriminate multiple roles of the gene products in the pathway, but with a minimum of the two major roles: activator and repressor of the pathway signalization.

There is thus provided according to an embodiment of the present invention, a method for analysis of the intracellular signaling pathway activation (SPA), the method including;
a. analyzing activator and repressor roles of a plurality of gene products in a plurality of pathways in at least one sample of at least one healthy subject and at least one sick subject to determine a pathway activation strength (PAS) for each of the plurality of pathways; and
b. comparing the pathway activation strength (PAS) in the at least one sick subject with the at least one healthy subject to determine intracellular signaling pathway activation (SPA) associated with a disease or disorder in the at least one sick subject.

There is thus provided according to an embodiment of the present invention, a computer software product, the product configured for analysis of the intracellular signaling pathway activation (SPA), the product including a computer-readable medium in which program instructions are stored, which instructions, when read by a computer, cause the computer to;
a. analyze activator and repressor roles of a plurality of gene products in a plurality of pathways in at least one sample of at least one healthy subject and at least one sick subject to determine a pathway activation strength (PAS) for each of the plurality of pathways; and
b. compare the pathway activation strength (PAS) in the at least one sick subject with the at least one healthy subject to determine intracellular signaling pathway activation (SPA) associated with a disease or disorder in the at least one sick subject.

There is thus provided according to an embodiment of the present invention, a system for analysis of the intracellular signaling pathway activation (SPA), the system including;
a. a processor adapted to activate a computer-readable medium in which program instructions are stored, which instructions, when read by a computer, cause the processor to;
   i. analyze activator and repressor roles of a plurality of gene products in a plurality of pathways in at least one sample of at least one healthy subject and at least one sick subject to determine a pathway activation strength (PAS) for each of the plurality of pathways; and
   ii. compare the pathway activation strength (PAS) in the at least one sick subject with the at least one healthy subject to determine intracellular signaling pathway activation (SPA) associated with a disease or disorder in the at least one sick subject.
b. a memory for storing the pathway activation strength (PAS) for each of the plurality of pathway and intracellular signaling pathway activation (SPA) associated with a disease or disorder; and
c. a display for displaying data associated with the at least one sick subject.

There is thus provided according to another embodiment of the present invention, a computer software product, the product configured for determining pathway activation in a biological sample, the product comprising a computer-readable medium in which program instructions are stored, which instructions, when read by a computer, cause the computer to:
a. determine for a biological pathway at least one concentration of a plurality of gene products involved in the pathway of the biological sample; and
b. compare the at least one concentration of the plurality of the gene product of the biological sample with a control set of concentrations of same gene products of at least one control sample to determine the pathway activation in the biological sample.

It is an object of some aspects of the present invention, to provide a proliferative transcriptome in which the transcribed genes in subjects with a proliferative disease or disorder relative (sick subjects) to healthy individuals are compared to define a set first of genes which are more strongly expressed (activated) in sick people or subjects relative to healthy people or subjects and a second set of genes (repressed) which are less strongly expressed in sick people or subjects relative to healthy people or subjects.

In some embodiments of the present invention, improved methods and software are provided for determining a pathway activation strength in sick subjects relative to healthy subjects of the same species. In some embodiments, the species is a vertebrate species. In some embodiments, the species is a mammalian species. In some preferred embodiments, the subject is a human species.

The generic cancer-protector rating approach involves collecting the transcriptome datasets from sick and healthy patients and normalizing the data for each cell and tissue type, evaluating the pathway activation strength (PAS) for each individual pathway and constructing the pathway cloud (PC 132, FIG. 1) and screen for drugs or combinations that minimize the signaling pathway cloud disturbance (Buzdin et al., 2014)) by acting on one or multiple elements of the pathway cloud. Drugs and combinations may be rated by their ability to return the signaling pathway activation pattern closer to that of the healthier tissue samples. The predictions may be then tested both in vitro and in vivo on human cells and on model organisms such as rodents, nematodes and flies to validate the screening and rating algorithms.

The present invention provides a method for ranking cancer-protective/treatment drugs, the method including collecting healthy subject transcriptome data and sick subject transcriptome data for one species to evaluate pathway activation strength (PAS) and down-regulation strength for a plurality of biological pathways, mapping the plurality of biological pathways for the activation strength and down-regulation strength from sick subject samples relative to healthy subject samples to form a pathway cloud map and providing a cancer-protective rating for each of a plurality of drugs in accordance with a drug rating for minimizing signaling pathway cloud disturbance (SPCD) in the pathway cloud map of the one species to provide a ranking of the cancer-protective drugs.

The present invention provides a new bioinformatic method, “OncoFinder™”, for both quantitative and qualitative analysis of the intracellular signaling pathway activation (SPA). This method is universal and may be used for the analysis of any physiological, stress, malignancy and other perturbed conditions at the molecular level. In contrast to the other existing techniques for aggregation and generalization of the gene expression data for individual samples, we suggest to distinguish the positive/activator and negative/repressor role of every gene product in each pathway. It is shown that the relative importance of each gene product in a pathway can be assessed using kinetic models for “low-level” protein interactions.

Although the importance factors for the pathway members cannot be so far established for most of the signaling pathways due to the lack of the required experimental data, we showed that ignoring these factors can be sometimes acceptable and that the simplified formula for SPA evaluation may be applied for many cases. We hope that due to its universal applicability, the OncoFinder method will be widely used by the researcher community.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic illustration of a system for analysis of intracellular signaling pathway activation using transcriptomic data, in accordance with an embodiment of the present invention;

FIG. 2 is a graph of values of pathway activation strength (APAS) that were calculated using the 98 random trials, each having random log-normally distributed weighting factors wn, versus non-perturbed PAS for the different SPs, calculated using OncoFinder method, in accordance with an embodiment of the present invention;

FIG. 3 is a simplified flow chart of a method for analysis of intracellular signaling pathway activation using transcriptomic data, in accordance with an embodiment of the present invention; and

FIGS. 4A and 4B are a simplified schematic display of a pathway cloud display, in accordance with an embodiment of the present invention.

In all the figures similar reference numerals identify similar parts.

### DETAILED DESCRIPTION OF PREFERRED EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

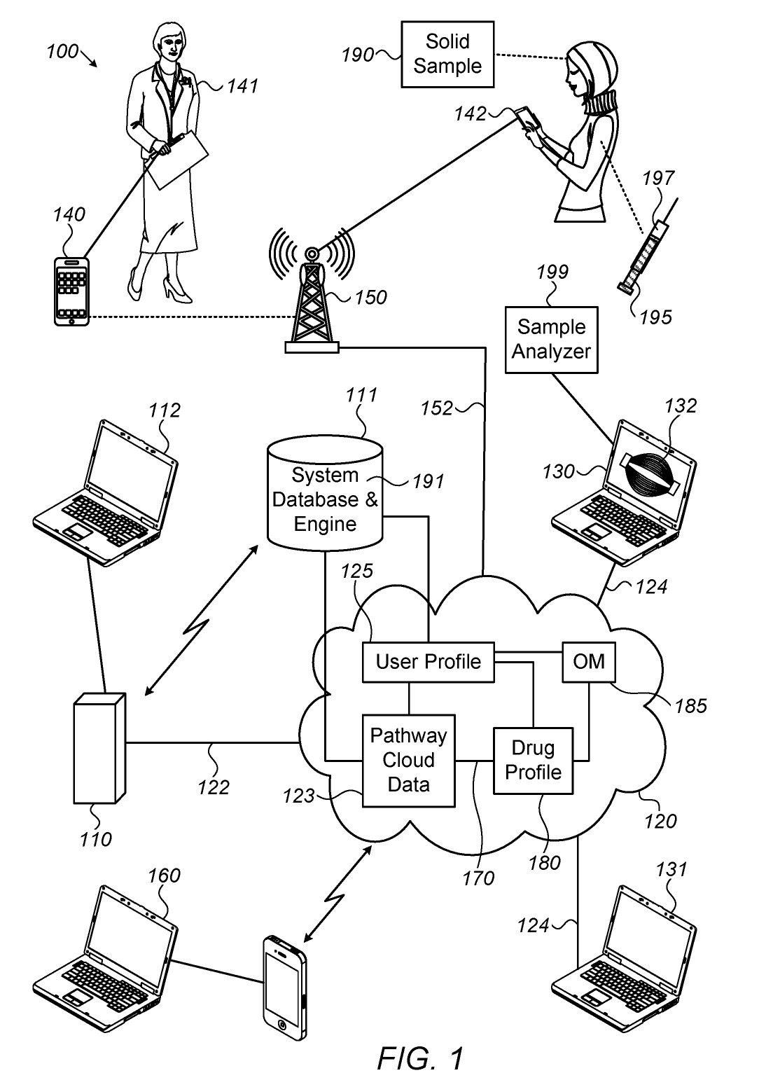

Reference is now made to FIG. 1, which is a simplified schematic illustration of a system for analysis of intracellular signaling pathway activation using transcriptomic data, in accordance with an embodiment of the present invention.

System 100 typically includes a server utility 110, which may include one or a plurality of servers and one or more control computer terminals 112 for programming, trouble-shooting servicing and other functions. Server utility 110 includes a system engine 111 and database, 191. Database 191 comprises a user profile database 125, a pathway cloud database 123 and a drug profile database 180.

Depending on the capabilities of a mobile device, system 100 may also be incorporated on a mobile device that synchronizes data with a cloud-based platform.

The drug profile database comprises data relating to a large number of drugs for controlling and treating cancer. For each type of drug, the dosage values, pharmo-kinetic data and profile, pharmodynamic data and profiles are included.

The drug profile database further comprises data of drug combinations, including dosage values pharmo-kinetic data and profile, pharmodynamic data and profiles.

A medical professional, research personnel or patient assistant/helper/carer 141 is connected via his/her mobile device 140 to server utility 110. The patient, subject or child 143 is also connected via his/her mobile device 142 to server utility 110. In some cases, the subject may be a mammalian subject, such as a mouse, rat, hamster, monkey, cat or dog, used in research and development. In other cases, the subject may be a vertebrate subject, such as a frog, fish or lizard. The patient or child is monitored using a sample analyzer 199. Sample analyzer 199, may be associated with one or more computers 130 and with server utility 110. Computer 130 and/or sample analyzer 199 may have software therein for performing the “Oncofinder method” of the present invention. The outputs of the software may be displayed, such as a cloud map 132, described in further detail hereinbelow and in Buzdin et al., 2014.

Typically, pathway cloud data 123 (FIG. 1), generated by the software of the present invention, is stored locally and/or in cloud 120 and/or on server 110.

The sample analyzer may be constructed and configured to receive a solid sample 190, such as a biopsy, a hair sample or other solid sample from patient 143, and/or a liquid sample 195, such as, but not limited to, urine, blood or saliva sample. The sample may be extracted by any suitable means, such as by a syringe 197.

The patient, subject or child 143 may be provided with a drug (not shown) by health professional/research/doctor 141.

System 100 further comprises an outputting module 185 for outputting data from the database via tweets, emails, voicemails and computer-generated spoken messages to the user, carers or doctors, via the Internet 120 (constituting a computer network), SMS, Instant Messaging, Fax through link 122.

Users, patients, health care professionals or customers 141, 143 may communicate with server 110 through a plurality of user computers 130, 131, or user devices 140, 142, which may be mainframe computers with terminals that permit individual to access a network, personal computers, portable computers, small hand-held computers and other, that are linked to the Internet 120 through a plurality of links 124. The Internet link of each of computers 130, 131, may be direct through a landline or a wireless line, or may be indirect, for example through an intranet that is linked through an appropriate server to the Internet. System 100 may also operate through communication protocols between computers over the Internet which technique is known to a person versed in the art and will not be elaborated herein.

Users may also communicate with the system through portable communication devices such as mobile phones 140, communicating with the Internet through a corresponding communication system (e.g. cellular system) 150 connectable to the Internet through link 152. As will readily be appreciated, this is a very simplified description, although the details should be clear to the artisan. Also, it should be noted that the invention is not limited to the user-associated communication devices—computers and portable and mobile communication devices—and a variety of others such as an interactive television system may also be used.

The system 100 also typically includes at least one call and/or user support and/or tele-health center 160. The service center typically provides both on-line and off-line services to users. The server system 110 is configured according to the invention to carry out the methods of the present invention described herein.

It should be understood that many variations to system 100 are envisaged, and this embodiment should not be construed as limiting. For example, a facsimile system or a phone device (wired telephone or mobile phone) may be designed to be connectable to a computer network (e.g. the Internet). Interactive televisions may be used for inputting and receiving data from the Internet. Future devices for communications via new communication networks are also deemed to be part of system 100. Memories may be on a physical server and/or in a virtual cloud.

A mobile computing device may also embody a non-synced or offline copy of memories, copies of pathway cloud data, user profiles database, drug profiles database and execute the system, engine locally.

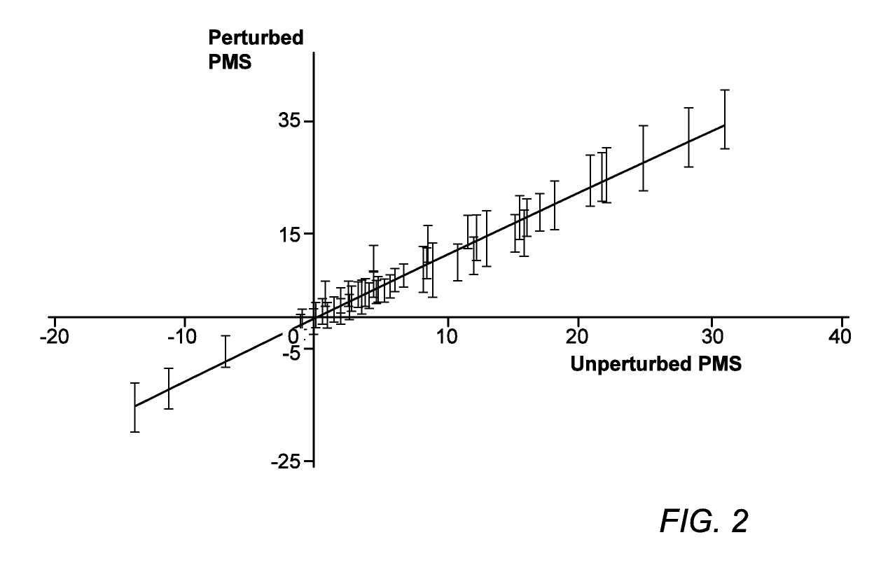

Reference is now made to FIG. 2, which is a graph of values of pathway activation strength (APAS), which were calculated using the 98 random trials, each having random log-normally distributed weighting factors wn, versus non-perturbed PAS for the different SPs, calculated using OncoFinder method. The pathway information was extracted from the SABiosciences database. Primary data are shown on the Supplementary dataset 3. For the perturbed values (APAS), both average values (points at the plot) and standard deviation bars are shown, in accordance with an embodiment of the present invention;

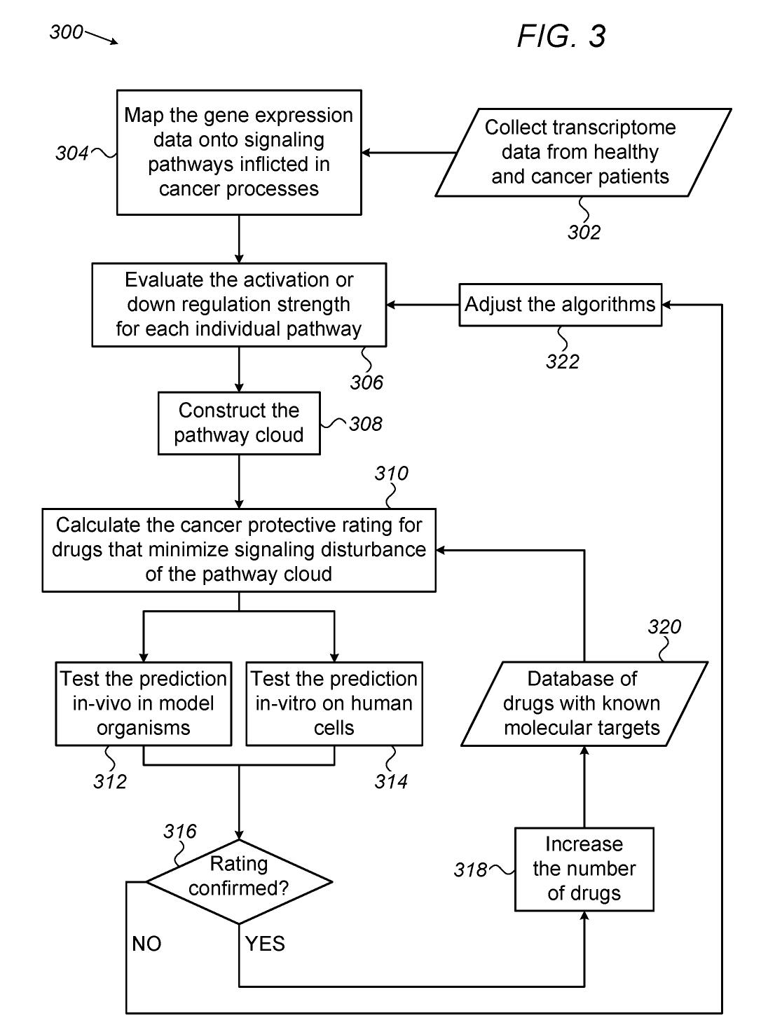

Reference is now made to FIG. 3, which is a simplified flow chart 300 of a method for analysis of intracellular signaling pathway activation using transcriptomic data, in accordance with an embodiment of the present invention.

In a first collecting step 302, transcriptome data from healthy and sick patients is collected and stored in a suitable database.

In a mapping step 304, the gene expression data collected in step 302 is mapped onto signaling pathways, which are affected by cancer processes.

In an evaluating step 306, the activation or down-regulation strength for each individual pathway is defined, providing one line per pathway. at least one algorithm may be as defined in Buzdin et al., (2014) below and may include at least one of:

$$a ) {PAS}_{p} = {\sum }_{n} {ARR}_{np} \cdot lg ( {CNR}_{n} ) ; \\ b ) {PAS}_{p}^{( 1 , 2 )} = {\sum }_{n} {ARR}_{np} \cdot {BTIF}_{n} \cdot {w}_{n}^{( 1 , 2 )} \cdot lg ( {CNR}_{n} ) ; and$$

$$S O = \frac{{\prod }_{i = 1}^{N} {[ AGEL ]}_{i}}{{\prod }_{j = 1}^{M} {[ RGEL ]}_{j}} ,$$

in order to define considers signal transducer protein of each pathway as having equal opportunities cause the activation/inhibition of pathway. Under these assumptions, based on the law of mass action next assessment of pathological changes in the signal pathway (signal outcome, SO).

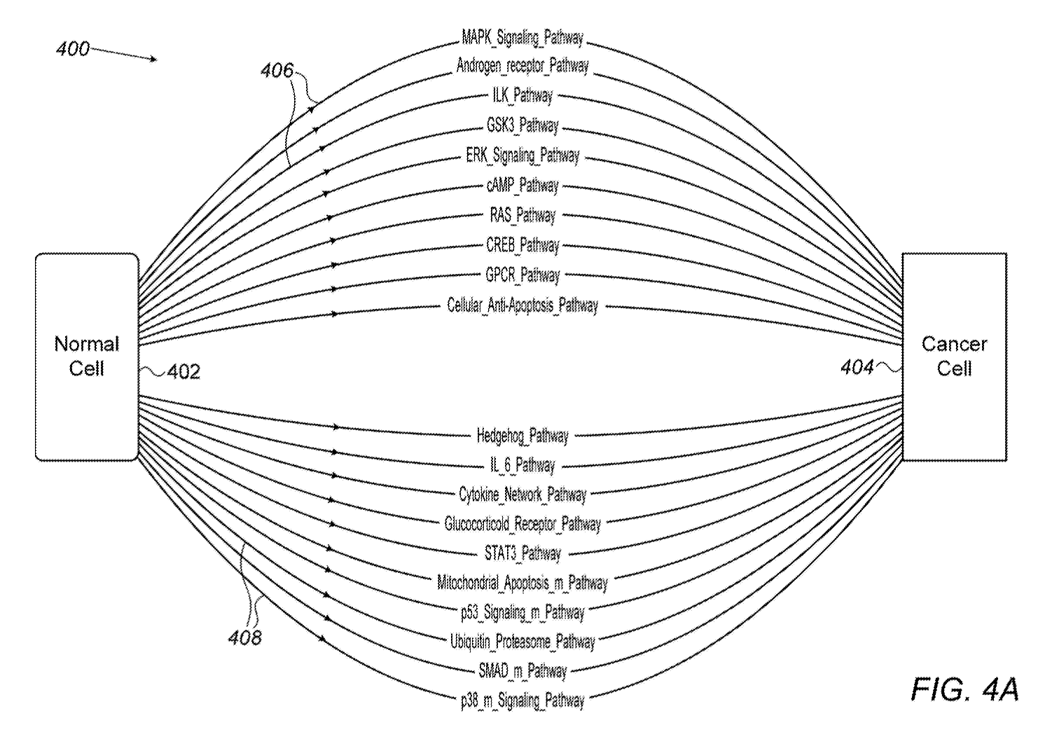

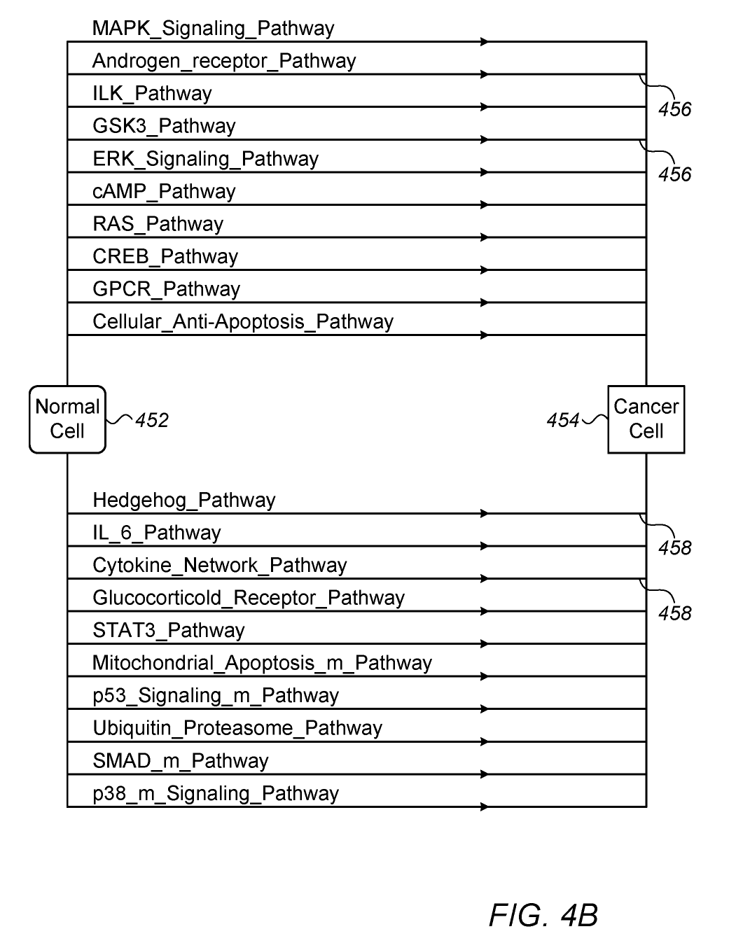

Thereafter, in a cloud construction step 308, a cloud for sick versus healthy and/or healthy versus sick is constructed. One non-limiting example of a cloud is shown in FIG. 4. Thus sets of upregulated and down-regulated paths are seen in 132, FIG. 1 and in further detail in FIG. 4, hereinbelow.

Thereafter for a number of drugs, the onco-protective rating of each drug which minimizes the signaling disturbance of the pathway cloud is determined in a gero-protective rating calculation step 310.

In one or more testing steps, 312, 314, the prediction of step 310 is tested in vivo in laboratory animals and in human species, respectively. The outputs of steps 312, 314 are testing data confirming the ratings of step 310.

A checking step 316 is performed to compare the ratings of step 310 to actual testing data.

If the testing data confirms the rating of step 310, a new drug is added to in an adding drug step 318. The data associated with the new drug is added to a database of drugs with known molecular targets in adding new drug step 318. Its potency to provide cancer treatment and/or onco-protection is calculated in step 310 and it is then tested in steps 312-314. Step 314 is repeated and then, according to its results, steps 316-318 or 320, 306-314 again.

If the testing data does not match the predicted rating from step 310, the algorithm is adjusted in adjusting step 322 and steps 306-314 are repeated for that drug.

Using the methodology of the present invention, one or more drugs can be defined that provide the best predicted outcomes for a certain patient, based on his/her phenotypic profile.

Moreover, using the methodology of the present invention, one or more drugs can be defined that provide the best predicted outcomes for a group of patients suffering from the same disease.

Additionally, the methods of the present invention may allow the use of one or more drugs, which provide the best predicted outcomes for a group of patients of the same ethnicity, suffering from the same disease.

FIG. 4A is a simplified schematic display of a pathway cloud display 400, in accordance with an embodiment of the present invention. The pathway cloud shows a normal cell or cell sample 402 and a cancer cell or cell sample 404. Between 402 and 404, are upregulated pathways 406 and down-regulated pathways 408. Thus, the schematic display shows the most and least altered signaling pathways, compared with the normal signaling pathways. Ten arrows on lines 406 in the upper part of the figure (top to bottom) are the ten most contributing to mitogenesis signaling pathways, ten arrows on lines 408 in the lower part of the figure are—the ten most hindering to mitogenesis signaling pathways. FIG. 4B is a simplified schematic display of a pathway cloud display 450, in accordance with an embodiment of the present invention.

The pathway cloud shows a normal cell or cell sample 452 and a cancer cell 454 or cell sample 454. Between 452 and 454, are upregulated pathways 456 and down-regulated pathways 458. Thus, the schematic display shows the most and least altered signaling pathways, compared with the normal signaling pathways. Ten arrows on lines 456 in the upper part of the figure (top to bottom) are the ten most contributing to mitogenesis signaling pathways, ten arrows on lines 458 in the lower part of the figure are—the ten most hindering to mitogenesis signaling pathways. The methodologies of the present invention make it possible to quantitatively estimate SPA for individual samples basing on the large-scale gene expression data. Theoretically, the signal transduction efficiency at every stage of the SP depends on the concentrations of the interacting gene products. The computational modeling of the signal transduction processes indicated that most of the interacting proteins can be found in the living cells at the concentrations significantly lower than the saturation levels for each transduction step (Bitwistle, 2007; Borisov, 2009).

The model of the present invention is based on the correlation of the signal transducer concentrations and the overall SPA. We also determined the overall individual roles of certain gene products in the functioning of each individual SP. These roles can be either positive or negative signal transduction regulators; alternatively, for some proteins the roles may be undefined or neutral. Finally, these roles may be characterized quantitatively depending on the individual importances of the individual interactors in the overall SPA.

The determination of these roles for each individual SP is a non-trivial task that has several uncertainties. Namely, protein interactions within each pathway may be competitive or independent, and therefore, belong to a sequential or parallel series of the nearby events (Borisov, 2006, Conzelman, 2006). The overall graph for the protein interaction events may include both sequential (pathway-like) and parallel (network-like) edges (Borisov, 2008; Conzelman, 2006). The role of each gene product in the signal transduction may depend on whether it works in a sequential or a parallel way.

Alternatively, as the raw approximation of this situation, one may propose a simplified method that utilizes only the overall roles of each gene product in the SPA.

In this case, each simplified signaling graph includes only two types of branches of protein interaction chain: one for sequential events that promote SPA, and another for repressor sequential events. Under these conditions, it can be presumed that all activator/repressor members have equal importances for the SPA, and come to the following formula for the overall signal outcome (SO) of a given pathway,

$$S O = \frac{{\prod }_{i = 1}^{N} {[ AGEL ]}_{i}}{{\prod }_{j = 1}^{M} {[ RGEL ]}_{j}} .$$

Here the multiplication is done over all possible activator and repressor proteins in the pathway, [AGEL]ᵢ and [RGEL]ⱼ are relative gene expression levels of activator (i) and repressor (j) members, respectively. To obtain an additive value, it is possible to take the logarithmic levels of gene expression, and thus come to a function of pathway activation strength, PAS, which operates with the experimental datasets obtained during comprehensive profiling of gene expression, for a pathway p,

$${PAS}_{p} = {\sum }_{n} {ARR}_{np} \cdot lg ( {CNR}_{n} ) .$$

Here the case-to-norm ratio, CNRₙ, is the ratio of the expression levels of a gene n in the sample (e.g., of a cancer patient) and in the control (e.g., average value for healthy group). The discrete value ARR (activator/repressor role) shows whether the gene product promotes SPA (1), inhibits it (−1) or plays an intermediate role (0.5, 0 or −0.5, respectively). Negative and positive overall PAS values correspond, respectively, to decreased or increased activity of SP in a sample, with the extent of this activity proportional to the absolute value of PAS.

However, the assumption of sequential protein-protein interaction in pathways may seem rather artificial. Although it is difficult to precisely estimate the importance of certain gene products that act in the pathway in a non-sequential mode, the solution may come from the kinetic models of SPA that use the “low-level” approach of mass action law describing each act of protein interactions. Some of these models were previously experimentally validated by us and others using Western blot analysis (Kholodenko, 1999; Kiyatkin, 2006; Bitwistle, 2007; Borisov, 2009; Kuzmina, 2011). Our previous experience suggests that the two models are especially effective for this task. One of them operates with the concept of sensitivity of the ordinary differential equation system with the free parameters (Kholodenko, 2003), which is generally applied to kinetic constants, but may be used for aspirating with the protein concentrations in the kinetic model of a pathway (Kuzmina, 2011), according to a formula,

$${w}_{j}^{( 1 )} = {lim}_{t \to ∞} \frac{1}{T} {\int }_{0}^{T} \vert \frac{\partial ln [ EFF ( t ) ]}{\partial ln {C}_{j}^{tot}} \vert dt .$$

Here w is the importance factor, [EFF(t)] is the time-dependent concentration of the active pathway effector protein (experimentally traced marker of a pathway activation), and Cⱼ^{tot} is the total concentration for the protein j.

Another way to calculate the importance factor for the gene products deals with the stiffness/sloppiness analysis of the effector activation (Daniels, 2008). This approach comprises analyzing the Hesse matrix,

$${H}_{ij} = \frac{{\partial }^{2}}{\partial {C}_{i}^{tot} \partial {C}_{j}^{tot}} {\sum }_{k} \frac{{( [ EFF ( {C}^{tot} , {t}_{k} ) ] - {[ EFF ]}_{k}^{exp} )}^{2}}{{σ}_{k}^{2}} ,$$

where C^{tot} is the vector of total concentrations for every protein in the pathway, [EFF(C^{tot},tₖ)] is concentration of an active pathway effector protein at the time point tₖ, [EFF]ₖ^{exp} is the experimentally measured (e.g., by Western blots) total concentration of the effector at the same time, and σₖ is the experimental error for this measurement. The sloppiness/stiffness analysis looks for the eigenvalues, □ₘ, and eigenvectors, □ₙ, for the Hesse matrix, Hξₘ=λₘ·ξₘ. The higher is the absolute value of □ₙ, the “stiffer” is the direction within the n-dimensional space of C^{tot} (where n is the number of protein types in the pathway model). The eigenvector components along with the stiffest direction, □ₛ, may be used for assessment of the importance factor w of a certain gene products in a pathway according to the formula: wⱼ⁽²⁾=|ξₛⱼ|.

Taking into account the above considerations, we come to the following final formula for assessing the SPA:

$${PAS}_{p}^{( 1 , 2 )} = {\sum }_{n} {ARR}_{np} \cdot {BTIF}_{n} \cdot {w}_{n}^{( 1 , 2 )} \cdot lg ( {CNR}_{n} ) .$$

Here the Boolean flag BTIF (beyond tolerance interval flag) indicates that the expression level for the gene n for the given sample is different enough from the respective expression level in the reference sample or set of reference samples. For this demonstration of our method we applied two simultaneous restriction/inclusion criteria to the expression of each individual gene: (i) 50% expression level cut-off rate compared to the average for the reference set, and (ii) the sample expression level should differ stronger than two standard deviations from the average of the reference set.

We next explored the effect of the introduction of the importance factors w in calculating PAS compared to the simplified model of PAS evaluation lacking w. Importance factors were calculated using either sensitivity-based, w⁽¹⁾, or stiffness-based, w⁽²⁾, algorithms. We performed this verification for the EGFR pathway, for which we established and published this model previously (Kuzmina, 2011). For these two sets of the importance factors, and for the w-free model, we performed a computational analysis of nine transcriptomes established using microarray hybridization technology for human glioblastoma samples from the published datasets (Supplementary dataset 1). The information on SP organization was taken from the Web-based SABiosciences database. The data on ARR were manually curated by analyzing the same database. Our findings suggest that the cloud of values for the ratio

$$\frac{{PAS}_{EGFR}^{( 1 )}}{{PAS}_{EGFR}}$$

(where PAS_{EGFR} is the PAS value for the EGFR pathway in the simplified model, where all importance factors equal to 1) lies within the interval of (0.6±0.8), whereas the ratio

$$\frac{{PAS}_{EGFR 1}^{( 2 )}}{{PAS}_{EGFR 1}}$$

belonged to the interval (1.0±0.8). Overall, we conclude that for such a complex SP like EGFR which includes >300 gene products, incorporation of the importance factors had only a moderate effect on the PAS. This suggests that, in principle, the simplified formula for PAS calculation may be applied for the pathway analysis.

For the overwhelming majority of the SPs, there is no experimental data available that makes impossible for them to calculate the importance factors using kinetic models. For them we performed the stochastic robustness analysis using the simplified formula for PAS. We introduced the additional random perturbation factor, wₙ, which was used as the analog of importance factor for PAS evaluation. In our computational simulation, the distribution of wₙ was logarithmically normal and calculated as follows: wₙ=2^{x}^{ₙ}, where xₙ were normally distributed random numbers with the expected value of M=0 and standard deviation σ=0.5. The random perturbation factors wₙ were applied to the glioblastoma transcriptional dataset GSM215422. Importantly, although the perturbation was done independently 98 times with independent weighting factors wₙ, for each gene, the values of standard deviation for the set of alternate PAS (APAS) were nor big enough to mask the proportional trend between the average perturbed PAS and unperturbed PAS for each of the 68 signaling pathways analyzed in this study (FIG. 1; Supplementary dataset 2).

We propose here a new biomathematical method, OncoFinder™, for both quantitative and qualitative analysis of the intracellular signaling pathway activation. It can be used for the analysis of any physiological, stress, malignancy and other perturbed conditions at the molecular level. The enclosed mathematical algorithm enables processing of high-throughput transcriptomic data, but there is no technical limitation to apply OncoFinder to the proteomic datasets as well, when the developments in proteomics allow generating proteome-wide expression datasets. We hope that due to its universal applicability, the method, OncoFinder, will be widely used by the biomedical researcher community and by all those interested in thorough characterization of the molecular events in the living cells. We also want to encourage building international scientific partnership aimed at the standardized experimental characterization of the importance factors for individual proteins, starting at least with the SPs most relevant to the major aspects of human physiology.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

Bauer-Mehren, A., Furlong, L. I., Sanz, F. (2009). Pathway databases and tools for their exploitation: benefits, current limitations and challenges. Mol Syst Biol, 5, article 290. doi: 10.1038/msb.2009.47.
Birtwistle, M. R., Hatakeyama, M., Yumoto, N., Ogunnaike, B. A. et al. (2007). Ligand-dependent responses of the ErbB signaling network: experimental and modeling analyses. Mol Syst Biol, 3, article 144. doi: 10.1038/msb4100188.
Borisov, N., Aksamitiene, E., Kiyatkin, A., Legewie, S. et al. (2009). Systems-level interactions between insulin-EGF networks amplify mitogenic signaling. Mol Syst Biol, 5, article 256, 2009. doi: 10.1038/msb.2009.19.
Borisov, N. M., Chistopolsky, A. S., Faeder, J. R., Kholodenko, B. N. (2008). Domain-oriented reduction of rule-based network models. IET Syst Biol, 2, 342-351. doi: 10.1049/iet-syb:20070081.
Borisov, N. M., Markevich, N. I., Hoek, L B., Kholodenko, B. N. (2006). Trading the micro-world of combinatorial complexity for the macro-world of protein interaction domains. BioSystems, 83, 152-166. doi: 10.1016/j.biosystems.2005.03.006.
Buzdin, Anton A., et al. “Oncofinder, a new method for the analysis of intracellular signaling pathway activation using transcriptomic data.” Frontiers in genetics 5 (2014).
Conzelmann, H., Saez-Rodriguez, J., Sauter, T., Kholodenko, B. N., Gilles E. D. (2006). A domain-oriented approach to the reduction of combinatorial complexity in signal transduction networks. BMC Bioinformatics, 7, article 4. doi:10.1186/1471-2105-7-34.
Daniels, B. C., Chen, Y. J., Sethna, J. P., Gutenkunst, R. N., Myers, C. R. (2008). Sloppiness, robustness and evolvability in systems biology. Curr Opin Biotechnol, 19, 389-395. arXiv:0805.2628v1
Elkon, R., Vesterman, R., Amit, N. (2008). SPIKE—a database, visualization and analysis tool of cellular signaling pathways. BMC Bioinformatics, 9, article 110: doi: 10.1093/nar/gkq1167.
GEO repository URL: http://www.ncbi.nlm.nih.gov/geo/Haw,
R., Stein, L. (2012). Using the Reactome database. Curr Protoc Bioinformatics, June, chapter 8, unit 8.7. doi: 10.1002/0471250953.bi0807s38.
Kholodenko, B. N., Demin, O. V., Moehren, G., Hoek, J. B. (1999). Quantification of short term signaling by the epidermal growth factor receptor. J Biol Chem, 274, 30169-30181. doi: 10.1074/jbc.274.42.30169.
Kholodenko, B., Kiyatkin, A., Bruggeman, F., Sontag, E., et al. (2003). Untangling the wires: a strategy to trace functional interactions in signaling and gene networks, Proc Natl Acad Sci, 20, 12841-12846. doi:10.1073/pnas.192442699.
Kiyatkin, A., Aksamitiene, E., Markevich N. I., Borisov, N. M. et al. (2006). Scaffolding protein GAB1 sustains epidermal growth factor-induced mitogenic and survival signaling by multiple positive feedback loops. J Biol Chem, 281, 19925-19938. doi: 10.1074/jbc.M600482200.
Kuzmina, N. B., Borisov, N. M. Handling complex rule-based models of mitogenic cell signaling (On the example of ERK activation upon EGF stimulation). (2011). Intl Proc Chem Biol Envir Engng, 5, 76-82.
Mathivanan, S., Periaswamy, B., Gandhi, T., Kandasamy, K. et al. (2006). An evaluation of human protein-protein interaction data in the public domain. BMC Bioinformatics, 7, article S19. doi:10.1186/1471-2105-7-S5-S19.
Nakaya, A., Katayama, T., Itoh, M., Hiranuka, K. et al. (2013). KEGG OC: a large-scale automatic construction of taxonomy-based ortholog clusters. Nucleic Acids Res, Jan, 41. doi: 10.1093/nar/gks1239.
Nikitin, A., Egorov, S., Daraselia, N., Mazo, I. (2003). Pathway studio—the analysis and navigation of molecular networks. Bioinformatics, 19, 2155-2157. doi: 10.1093/bioinformatics/btg290.
SABiosciences, a Qiagen company. URL: http://www.sabiosciences.com/pathwaycentral.php
The UniProt consortium. (2011). Ongoing and future developments at the Universal Protein Resource. Nucleic Acids Research, 39, D214-D219. doi: 10.1093/nar/gkq1020.
Yizhak K., Gabay O., Cohen H., Rupin E. (2013). Model-based identification of drug targets that revert disrupted metabolism and its application to ageing. Nature Communications, 4, 2632-doi: 10.1038/ncomms3632.

1. A method for determining pathway activation in a biological sample from a mammalian subject, the method comprising:
a. determining for a biological pathway at least one concentration of a plurality of gene products involved in said pathway of said biological sample;
b. comparing said at least one concentration of said plurality of said gene product of said biological sample with a control set of concentrations of same gene products of at least one control sample to determine said pathway activation in said biological sample; and
c. calculating a pathway activation strength (PAS), indicative of said pathway activation of said biological pathway, wherein said calculating step comprises adding concentrations of said set of said plurality of gene products of said sample and comparing to a same set in said at least one control sample.

2. A method according to claim 1, wherein said biological sample is selected from the group consisting of a tissue sample, a cell culture, an individual single cell, a bodily sample, an organism sample and a microorganism sample.

3. A method according to claim 1, wherein said pathway activation is a biological pathway activation.

4. A method according to claim 3, wherein said biological pathway is a signaling pathway.

5. A method according to claim 3, wherein said biological pathway is a metabolic pathway.

6. A method according to claim 1, wherein said plurality of gene products comprises a set of at least five gene products.

7. (canceled)

8. (canceled)

9. A method according to claim 1, wherein said plurality of gene products provides at least one function in said biological pathway.

10. A method according to claim 9, wherein said at least one function comprises an activation function and a suppressor function.

11. A method according to claim 9, wherein said at least one function comprises an up-regulating function and a down-regulating function.

12. A method according to claim 9, wherein said method further comprises determining an overall signal outcome (SO).

13. A method according to claim 12, wherein said signal outcome (SO) is defined as
$$S O = \frac{{\prod }_{i = 1}^{N} {[ AGEL ]}_{i}}{{\prod }_{j = 1}^{M} {[ RGEL ]}_{j}} ,$$
and wherein multiplication is performed for possible activator and repressor proteins in said pathway, and [AGEL]ᵢ and [RGEL]ⱼ are relative gene expression levels of activator (i) and repressor (j) members, respectively.

14. A method according to claim 1, wherein said determining step comprises at least one of profiling gene expression, RNA profiling, RNA sequencing, DNA profiling, DNA sequencing, protein profiling, amino acid sequencing, at least one immunochemical methodology, a mass spectrometry analysis, a microarray technology, a quantitative PCR methodology and combinations thereof.

15. A method according to claim 14, wherein said method is quantitative.

16. A method according to claim 14, wherein said method is qualitative.

17. (canceled)

18. (canceled)

19. A method according to claim 1, wherein said mammalian subject is human.

20. A method according to claim 19, wherein said subject is sick.

21. A method according to claim 20, wherein said sick subject suffers from a proliferative disease or disorder.

22. A method according to claim 21, wherein said proliferative disease or disorder is cancer.

23. A method according to claim 1, wherein said PAS is defined by
$${PAS}_{p} = {\sum }_{n} {ARR}_{np} \cdot lg ( {CNR}_{n} ) .$$

24. A method according to claim 7, wherein a signaling pathway activation (SPA) is determined by
$${PAS}_{p}^{( 1 , 2 )} = {\sum }_{n} {ARR}_{np} \cdot {BTIF}_{n} \cdot {w}_{n}^{( 1 , 2 )} \cdot lg ( {CNR}_{n} ) .$$

25.-37. (canceled)
